(19) United States
(12) Patent Application Publication
(10) Pub. No.: US 2022/0087994 A1
(43) Pub. Date: Mar. 24, 2022

(54) HIGH LOAD DISPERSIONS AND USES THEREOF

(71) Applicant: Austin Research Labs Corp., Buffalo, NY (US)

(72) Inventors: Lauren McMillan, Buffalo, NY (US); Jai Supamahitorn, Buffalo, NY (US)

(21) Appl. No.: 17/423,198

(22) PCT Filed: Jan. 15, 2020
(86) PCT No.: PCT/US2020/013637

§ 371 Date: Jul. 15, 2021

Related U.S. Application Data
(60) Provisional application No. 62/792,976, filed on Jan. 16, 2019.

(51) Int. Cl. A61K 31/4425; A23K 50/10; A61K 47/46; A23K 20/111; A61P 1/12; A23K 20/28; A61K 45/06; A23K 20/132; A61K 9/00; A23K 50/60; A61K 31/14

(52) U.S. Cl. CPC A61K 31/4425; A23K 50/10; A61K 47/46; A23K 20/111; A61P 1/12; A61K 31/14; A61K 45/06; A23K 20/132; A61K 9/0056; A23K 50/60; A23K 20/28

(57) ABSTRACT

The present disclosure relates to high load dispersions and uses thereof. In particular, the present disclosure provides high load dispersions for use in preventing and treating disease in calves, cattle, and other animals.

This application claims priority to U.S. provisional patent application Ser. No. 62/792,976, filed Jan. 16, 2019, which is incorporated herein by reference in its entirety.

### FIELD OF THE DISCLOSURE

The present disclosure relates to high load dispersions and uses thereof. In particular, the present disclosure provides high load dispersions for use in preventing and treating disease in calves, cattle, and other animals.

### BACKGROUND OF THE DISCLOSURE

Scours is a significant gastrointestinal infectious disease that results in diarrhea and the disease enteritis, meaning inflammation of the intestinal tract. The incidence of scours in neonatal calves averages 30% year-round and has been reported to be in excess of 50% during the winter months. Most cases of scours occur in the newborn calf within one month of age, with the greatest majority occurring between 3 and 14 days of life. The bacterial burden of scours on the calf results in dehydration and inflammation of the intestinal walls, thus impairing the calf's ability to absorb nutrients and put on weight at an appropriate rate. In many instances, the presence of bacteria and the toxins they release can harm vital organs and even result in death. The occurrence of scours remains a great burden to the dairy and beef cattle industries given the morbidity, mortality, time and financial costs associated with this disease.

Additional therapies for scours are needed.

### SUMMARY OF THE DISCLOSURE

The present disclosure relates to high load dispersions and uses thereof. In particular, the present disclosure provides high load dispersions for use in preventing and treating disease in calves, cattle, and other animals.

For example, in some embodiments, provided herein is a method of treating or preventing enteritis and/or diarrhea in an animal (e.g., cattle), comprising: administering a dispersion to said cattle, the dispersion comprising a dispersed phase within an aqueous phase, and wherein the dispersion comprises: a) a hydrophilic clay in the dispersed phase; and b) a quaternary ammonium compound in the dispersed phase, wherein the administering treats or prevents said enteritis and/or diarrhea in the animal. In some embodiments, the enteritis and/or diarrhea is caused by a bacterial infection. In some embodiments, the administration kills or prevent the growth of the bacteria and/or neutralizes a toxin secreted by the bacteria. In some embodiments, the administering prevents death of the cattle. In some embodiments, the administering treats or prevents sepsis (e.g., sepsis caused by bacteria related to the enteritis and/or diarrhea).

Further embodiments provide the use of a composition comprising a dispersion, the dispersion comprising a dispersed phase within an aqueous phase, and wherein the dispersion comprises: a) a hydrophilic clay in the dispersed phase; and b) a quaternary ammonium compound in the dispersed phase, to treat or prevent enteritis and/or diarrhea in an animal (e.g., cattle).

In yet other embodiments, provided is a composition comprising a dispersion, the dispersion comprising a dispersed phase within an aqueous phase, and wherein the dispersion comprises: a) a hydrophilic clay in the dispersed phase; and b) a quaternary ammonium compound in the dispersed phase, for use to treat or prevent enteritis and/or diarrhea in an animal (e.g., cattle).

In some embodiments, the hydrophilic clay comprises one or more of smectite, laponite, hectorite, montmorillonite, or bentonite clays. In some embodiments, the quaternary ammonium compound comprises benzethonium chloride, cetylpyridinium chloride, or derivatives thereof. In some embodiments, the dispersion is combined with one or more of a milk replacer, water, or dry animal feed. In some embodiments, the dispersion is administered intravenously. In some embodiments, the dispersion further comprises an active agent (e.g., one or more of; an antimicrobial, an analgesic, an anti-fungal, or an anti-inflammatory). In some embodiments, the dispersion comprises one or more of a chelating agent, emulsifier, humectants, moisturizer, detackifier, emollient, animal feed, flavoring agent, or thickener.

In some embodiments, the cattle are calves. In some embodiments, the calves are 0 to 30 days old. In some embodiments, the administering is repeated daily for 1 to 30 days. In some embodiments, the administering is initiated within 2 to 12 hours after birth of the cattle. In some embodiments, the administering is at least 2 hours after an initial colostrum feeding. In some embodiments, the administering is at least 2 hours after feeding the cattle.

In some embodiments, the dispersion is made by the method of i) dry mixing the hydrophilic clay and the quaternary ammonium compound to produce a mixture; ii) adding water to the mixture; and iii) incubating the mixture to form the dispersion.

Further embodiments are described herein.

### BRIEF DESCRIPTION OF THE DRAWINGS

FIG. 1 shows a plot of symptoms of scours of calves treated with a dispersion of embodiments of the present disclosure (top) and control (bottom).

FIG. 2 show graphs of symptoms of scours of calves treated with a dispersion of embodiments of the present disclosure and control.

### DEFINITIONS

As used herein, the term “dispersion” refers to a stable suspension. Such dispersions may be stable due to particle size and/or the presence of components having both hydrophilic and hydrophobic sites, e.g., as in a surfactant or emulsifier. In some embodiments, the dispersion is stable for more than one day, one week, one month, etc.

As used herein, the terms “continuous phase” and “dispersed phase” are related to a dispersion system, in which a first material is dispersed within a second material fine solid or liquid particles. In such a dispersion system, the term “continuous phase” refers to a first phase surrounding a second “dispersed phase.” The “dispersed phase” refers to the suspended particles or liquid droplets dispersed in the continuous phase.

As used herein, the term “emulsion” refers to a heterogeneous system comprising a continuous phase and a non-continuous phase capable of forming droplets in the continuous phase.

As used herein, the term “emulsifier” refers to an agent that can reduce and/or eliminate the surface and the interfacial tension in a two-phase system. The emulsifier agent may possess both hydrophilic and lipophilic groups. The emulsifier may be considered to be either in the continuous phase, dispersed phase, or both.

As used herein, the phrase “in association with” is intended to include any or all of: chemical combination, charge attraction, entrapment, whole or partial dissolution, and suspension.

As used herein, the term “subject” refers to any animal (e.g., a mammal), including, but not limited to, humans, non-human primates, companion animals (e.g., dogs, cats, etc.), livestock (e.g., horses, cows, goats, sheep, pigs, etc.), rodents, birds, and the like, which is to be the recipient of a particular treatment. In some embodiments, the subject is a calf.

As used herein, the term “pharmaceutical composition” refers to the combination of an active agent with a carrier, inert or active, making the composition especially suitable for diagnostic or therapeutic use in vivo, or ex vivo.

As used herein, the term “toxic” refers to any detrimental or harmful effects on a cell or tissue as compared to the same cell or tissue prior to the administration of the toxicant.

### DETAILED DESCRIPTION OF THE DISCLOSURE

The present disclosure relates to high load dispersions and uses thereof. In particular, the present disclosure provides high load dispersions for use in preventing and treating disease in animals (e.g., calves and cattle).

Calf scours is a clinical sign associated with several diseases characterized by diarrhea and/or enteritis. Calf scours can be caused by noninfectious causes or infectious causes (e.g., bacterial (e.g., including but not limited to, Escherichia coli, Salmonella spp., or Clostridium perfringens), viral (e.g., rotavirus, coronavirus, BVD virus, or IBR virus), protozoan parasites (e.g., Cryptosporidium Coccidia), and yeasts and molds.

For example, in some embodiments, provided herein are dispersions for use in treating or preventing disease in animals (e.g., calf scours (e.g., calf scours caused by bacterial infection)). In some embodiments, the dispersion comprises a dispersed phase within a continuous or aqueous phase. In some embodiments, a continuous phase comprises a liquid or gel, and may optionally comprise one or more of a humectant, emollient, detackifier, moisturizer, thickener, chelating agent, or other additive. In some embodiments, a dispersed phase comprises: a hydrophilic particle substrate having electrically charged binding sites, an intermediate component comprising a hydrophobic moiety and an ionic moiety (e.g. electrically charged moiety, hydrophilic moiety), and optionally an active agent (e.g., biologically active agent (e.g., antimicrobial agent, etc.) comprising a hydrophobic moiety. In some embodiments, the ionic moiety of the intermediate component is attracted to the electrically charged binding sites of the hydrophilic particle substrate. In some embodiments, the hydrophobic moiety of the intermediate component is attracted to the hydrophobic moiety of the active agent. In some embodiments, attractive forces between the intermediate component and both the hydrophilic particle substrate and active agent cause the components to assemble into a complex or supramolecular particle.

Exemplary dispersions are described below.

### Dispersions

In some embodiments, compositions (e.g. dispersions) have viscosities of at least 100 centipoise (e.g., >100 centipoise, 100-150 centipoise, 150-200 centipoise, >200 centipoise, 200-300 centipoise, >300 centipoise, >500 centipoise). In some embodiments, compositions have viscosities of 150-200 centipoise. In some embodiments, compositions have viscosities of greater than about 1000 centipoise (e.g., 1000 centipoise . . . 2000 centipoise . . . 5000 centipoise . . . 10,000 centipoise . . . 20,000 centipoise . . . 50,000 centipoise . . . 100,000 centipoise . . . 200,000 centipoise, etc.). In some embodiments, compositions are gels. In some embodiments, desired viscosities are obtained by inclusion of gelling agents, high viscosity additives, and increase concentration of solids (e.g., >10%, >15%, >20%, >25%, >30%, >40%, >50%, etc.).

In some embodiments, compositions for intravenous or other administration have a viscosity of less than 10 centipoise.

In some embodiments, a dispersion has a continuous aqueous phase containing particulate material held in suspension by small particle size, e.g., sub-micron, or by an emulsifier. In some embodiments, a dispersion has a suspended phase including suspended microparticles or smaller and may include hydrophobic droplets. The droplets and sometimes solid particles, may be held in suspension with the assistance of an emulsifier. In some embodiments, “continuous aqueous phase” or “aqueous phase” refers to the continuous phase surrounding solid particle and/or hydrophobic droplets. The aqueous phase thus contains suspended or dissolved components, e.g., thickeners, gelling agents, humectants, moisturizers, emulsifiers, chelating agents, stabilizers, adherents, emollients, dyes and fragrances. Emollients and emulsifiers may be considered as being in an intermediate phases between aqueous (polar) and non-polar (oil) phases. The particles and hydrophobic droplets may be considered to be the suspended phase. The water used to form the aqueous phase is deionized water obtained by any known means, e.g., ion exchange resins or distillation in an inert system, e.g., in non-reactive glass, or reverse osmosis.

In some embodiments, the compounds used for the intermediate components comprise ligands, i.e., bound to the central particle and arranged to accept additional components at exposed portions. In some embodiments, compound for use in an intermediate layer is a quaternary ammonium compound having a hydrophobic tail; although, any other compound having an ionic structure may be used and attracted as above described.

Ligands having antimicrobial properties include compounds having reactive inorganic cations, particularly those which have one or more electrons available for chemical reactions (e.g., transition metals) and compounds containing organic cations known to have bactericidal activity. For example, the antimicrobial effects of quaternary ammonium compounds, iodophor compounds, phenolics, alcohol, chlorine, peroxides, aldehydes and metals have been well documented. For further detail, see U.S. Pat. No. 6,288,076, which is hereby incorporated by reference in its entirety. Ligands having antimicrobial properties which are particularly desirable for use as ligands in the present disclosure include quaternary ammonium compounds, transition metals, organo metallic compounds, perchlorates, charged halogen-containing compounds, charged organic peroxides, ionic polymers, ionic surfactants, and mixtures thereof.

Especially desirable quaternary ammonium compounds include hexadecyltrimethyl ammonium bromide, trimethylphenyl ammonium chloride, cetylpyridinium chloride, and mixtures thereof. Especially desirable transition metals include copper, iron, manganese, zinc, silver, mercury, and mixtures thereof. The antimicrobial agent of the present disclosure includes ligands attached to the colloidal particles in excess of and up to 200% of the C.E.C. of the colloid particles (e.g., greater than 250% or greater than 300%), resulting in greater safety and efficacy of the antimicrobial agent.

A preferred quaternary compound having cationic activity is benzethonium chloride. Benzethonium chloride, having an ionic hydrophilic site and a hydrophobic tail and being an antimicrobial and thus active may be used in both the secondary and tertiary layers. The benzethonium chloride may be present in an amount of, for example, 0.5% by weight of solids.

Quaternary ammonium compound may be used as a secondary layer and/or an antimicrobial tertiary layer, e.g., in an amount of, for example, 0.50% by weight of solids or at an amount per dose of 0.001 to 20 g.

When compounds have such properties, they may be directly and indirectly loaded onto the particle substrate in two layers. Complete loading on a single layer may be considered as a 100% loading and when complete loading occurs on both the intermediate secondary and tertiary layers, loading may be considered to be 200%. The present disclosure may permit loading as high as 200% or even higher due to additional complex interactions.

In some embodiments, the present disclosure provides particle substrates. In some embodiments, particle substrates comprise hydrophilic particle substrates. In some embodiments, particle substrates comprise hydrophilic submicorn particle substrates. In some embodiments, particle substrates comprise electrically charged binding sites. In some embodiments, particle substrates comprise hydrophilic sites (e.g., hydrophilic binding sites). In some embodiments, hydrophilic sites are due to ionic moieties, e.g., a quaternary, carboxy, sulfo, phosphor, or a polar component such as may be found in a chemically bound oxygen, nitrogen, sulfur or phosphorous atom having an exposed electron pair. Such components may be compounds or aggregations. Particular examples are anionic, cationic and non-ionic groups as may be found in surfactants. In some embodiments, particle substrates have submicron diameters (e.g., <1 μm, <0.5 μm, <0.2 μm, <0.1 μm, <0.05 μm, <0.02 μm, <0.01 μm, etc.). In some embodiments, submicron size provides stability. In some embodiments, submicron particles are nanoparticles that require no stabilization.

As used here, the term “particle substrate” means a particle that acts as a substrate for an interaction with an agent, compound, ligand, reactive group, functional group, etc. An example of particle substrates that find use in the present disclosure is a hydrophilic hydrated clay. Such clays are primarily aluminosilicates in which some of the aluminium and silicon ions have been replaced by elements with different valence, or charge. For example, aluminium (Al³⁺) may be replaced by iron (Fe²⁺) or magnesium (Mg²⁺), leading to a net negative charge. This charge attracts positive cations which in turn may attract a corresponding anion.

The particles may be organic and inorganic particles, including nano-particles. Preferred inorganic materials have surface areas ranging from 50-1000 m²/gm, with surface areas of 500-800 m²/gm being especially desirable. Useful synthetic types of clay-type minerals include a synthetic hectorite, which is a layered hydrous magnesium silicate, such as LAPONITE (BYK Additives & Instruments, Germany, formerly Southern Clay Products, Gonzales, Tex.), a synthetic mica-montmorillonite, such as BARASYM, (Baroid Division, NL Industries, Houston, Tex.) and mixtures thereof. Useful naturally occurring clay minerals include swelling clays such as aliettite, beidellite, bentonite, nontronite, saponite, sauconite, stevensite, swinefordite, volkonskoite, yakhontovite, hectorite, montmorillonite (such as BP colloid), and mixtures thereof. Other useful materials (both synthetic and naturally occurring) include, but are not limited to polymers, zeolites, layered double hydroxides, illite, chlorite, kaolinite, hydrotalcite, talc, halloysite, sepiolite, and palygorskite, as well as other minerals such as oxides, hydroxides, and silicates, to name just a few. Typically, the colloid particles of the present disclosure have a mean diameter of 1 nm to 100 microns, having mean diameters of less than 2 microns with diameters of less than one micron being preferred.

Preferred clays are hydrophilic smectite, laponite and bentonite clays having high cationic exchange properties. Other suitable particles are ion exchange resin particles, and organic plastic particles having charged sites.

In some embodiments, particles are characterized by both large surface areas and substantial ion exchange capacities. Such ion exchange capacities are usually, but not always cation exchange capacities (CEC). It is to be understood that anion exchange resins may also be used, e.g., polyfunctional resins containing quaternary amine groups. In general where “CEC” is used herein, it should be understood that anion exchange resins may also be used in the appropriate context. The number of binding sites on a particle may be determined by binding sites per mole when the structural formula of the resin is known as modified by surface characteristics, e.g., surface area due to particle sizes effects. A number of CEC's are known for particular materials, e.g., for laponites used in examples herein are known to have a CEC of about 55.0 meq/100 grams. Compositions provided herein are unique in that loadings well in excess of the CEC may be obtained, e.g., over 125% up to as much as 250% or more.

Bioactive compositions, made according to the methods of the present disclosure use a variety of substrates, examples of which are given below, in addition to a variety of bioactive compounds that are attached to the substrate. By varying the organics that are used for ion exchange to prepare the organo-substrate, the organo-substrate can be tailored to have either hydrophilic or hydrophobic surface tension properties. Furthermore, by choosing the appropriate carrier substrate, e.g., clay, that is used for additional attachment of organic onto the organo-substrate, the antimicrobials produced can exhibit either hydrophilic or hydrophobic properties. This allows the compositions to be used in either aqueous or non-aqueous formulations.

In certain embodiments, active agents comprise a tertiary layer of particulates of the present disclosure. In some embodiments, active agents (e.g., molecules for forming the tertiary layer) have a hydrophobic moiety (e.g., hydrophobic tail). In other embodiments, active agents (e.g., molecules for forming the tertiary layer) have a biologically active moiety. In particular embodiments, active agents are quaternary compounds. In some embodiments, active agents are antimicrobials, humectants, moisturizers, anti-inflammatory, and nutrients.

In some embodiments, a quaternary compound comprises one or more antimicrobial agents, including, but not limited to: lauryl dimethyl benzylammonium chloride, benzalkonium chloride, alkyltrimethyl ammonium chloride, dialkyldimethylammonium chloride, alkyldimethylbenzylammonium chloride, alkyldimethyl(ethylbenzyl)ammonium chloride, combinations thereof, etc. In other embodiments, a quaternary compound comprises one or more non-antimicrobial conditioning agents, including, but not limited to: cetrimide, cetrimonium bromide, cetylamidopropyldimethyl ammonium chloride, stearyl trimethyl ammonium chloride, stearalkonium chloride, dihydrogenated tallow dimethyl ammonium chloride, combinations thereof, etc.

Specific, non-limiting examples of suitable hydrophobic active ingredients are: acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. In addition, salts, isomers and derivatives of the above-listed hydrophobic active ingredients may also be used, as well as mixtures.

In some embodiments, the dispersion further comprises an active agent that is a drug (e.g., anti-inflammatory drug, analgesic, antibiotic, and the like).

In some embodiments, dispersions have antimicrobial activity without the addition of a further active agent or drug.

Dispersions may comprise additional compounds including, but not limited to, emulsifiers, chelating agents, gelling agents, stabilizers, adherents, emollients, dyes, detackifiers, thickeners, nonaqueous moisturizers, anti-inflammatory agents, and animal feed.

Dispersions may also comprise a detackifier such as phenyl substituted silicone fluid, e.g., phenyl trimethicone. In some embodiments, a detackifier also acts as a humectant. In some embodiments, dispersions comprise an emollient, e.g., pentaerythrityl tetracaprylate.

In certain embodiments, a dispersion comprises one or more dyes and/or pigments, including, but not limited to: titanium dioxide, natural mined and synthetic iron oxides, blends of inorganic oxides and fillers (kaolin, talc, silica, mica), D&C colors, FD&C colors, combinations thereof, etc. In some embodiments, a dispersion comprises one or more dyes, including, but not limited to: Disperse Red 13, Disperse Green 9, Solvent Black 3, Disperse Blue 148, Disperse Violet 63, Disperse Blue, Disperse Blue 14, Solvent Orange 15, Solvent Orange 7, Solvent Blue 14, Disperse Yellow 82, 9-diethylamino-5H-benzo[alpha]phenoxazine-5-one, 1-dimethylamino-5-sulfamoyl-naphthalene, pyrene, 1-pyrenecarbaldehyde, Reichardt's dye, 4-aminophthalimide, 4-(N,N-dimethylamino)phthalimide, bromonaphthalene, 2-(dimethylamino)naphthalene, solvatochromic dye, combinations thereof, etc.

In particular embodiments, a dispersion comprises one or more fragrances, including, but not limited to: tea tree oil, citrus oils (e.g., lemon oil, orange oil, etc.), oils from herbs (e.g., rosemary, oil, thyme oil, oregano oil, etc.), oils from woods (e.g., rosewood oil, cedarwood oil), cinnamaldehyde or cinnamon bark oil, eugenol or clove flower oil, cineol or eucalyptus oil, camphor or camphor tree oil, geraniol or palmarosa oil, citronella oil, geranium oil, cedrol, etc. In some embodiments, the present disclosure provides any suitable essential oil. In some embodiments, fragrances further provide antimicrobial, fungicidal, and/or insect-repelling functionality.

In some embodiments, a dispersion comprises one or more emulsifiers, including, but not limited to: PEG-dimethicones, polyglycerol dimethicones, Sorbian oleate, glyceryl esters, C12-15 alkyl benzoate, castor oil, cetearyl alcohol, cetyl alcohol, cetyl esters, cetyl palmitate, diisopropyl adipate, emu oil, isopropyl myristate, isopropyl palmitate, lanolin, Mangifera indica seed butter, mineral oil, myristyl myristate, sorbitan oleate, safflower oil, shea butter, stearic acid, stearyl alcohol, calcium stearoyl lactylate, ceteareth-20, cocamide MEA, glyceryl laurate, glyceryl stearate, glyceryl stearate and PEG-100 stearate, glyceryl stearate SE, glycol distearate, glycol stearate, isoceteth-20, isosteareth-20, lauramide DEA, laureth-23, laureth-4, linoleamide DEA, methyl glucose sesquistearate, oleth-10, oleth-10/polyoxyl 10 oleyl ether NF, Oleth-2, Oleth-20, PEG-100 Stearate, PEG-20 Almond Glycerides, PEG-20 Methyl Glucose Sesquistearate, PEG-25 Hydrogenated castor oil, PEG-30 dipolyhydroxystearate, PEG-4 dilaurate, PEG-40 sorbitan peroleate, PEG-60 almond glycerides, PEG-laurate, PEG-80 sorbitan laurate, polysorbate 20, polysorbate 60, polysorbate 80, polysorbate 85, sodium stearoyl lactylate, sorbitan isostearate, sorbitan laurate, sorbitan sesquioleate, sorbitan stearate, sorbitan stearate and sucrose cocoate, sorbitan trioleate, stearamide MEA, steareth-2, steareth-21, combinations thereof, etc.

Dispersion may comprise one or more humectants, including, but not limited to: polyglycerol dimethicones, gelatin, glycerin, honey, hyaluronic acid, panthenol, propylene glycol, sodium ammonium lactate, sodium pyrrolidine carboxylic acid, sorbitol, urea, 1,2,6 hexanetriol, Hexylene and Butylene Glycol, Dipropylene glycol, Hexylene Glycol, Panthenol, Phytantriol, Sodium PCA, Triethylene glycol, olyglyceryl sorbitol, Glucose, Fructose, Polydextrose, Potassium PCA, Hydrogenated Honey, Inositol, Hexanediol beeswax, Hexanetriol Beeswax, Hydrolyzed Elastin, Hydrolyzed Collagen, Hydrolyzed Silk, Hydrolyzed Keratin, Erythritol, Capryl glycol, Isoceteth-(3-10, 20, 30), Isolaureth-(3-10, 20, 30), Laneth-(5-50), Laureth-(1-30), Steareth-(4-20), Trideceth-(5-50), sucrose, glucose, aloe, alpha-hydroxy acids (AHA's), combinations thereof, etc.

In some embodiments, a dispersion comprises one or more thickeners and/or stabilizers, including, but not limited to: dimethicone gums, dimethicone cross-polymers, stearic acid, stearic acid with cetyl alcohol, cellulose, carbopol, polyacrylic acid, clays, carrageenan, pectin, and locust bean gum, xantham gum, carbomer (a homopolymer of acrylic acid with a high molecular weight, which is cross-linked with any of several polyalcohol allyl ethers), combinations thereof, etc.

In certain embodiments, a dispersion comprises one or more detackifiers and/or emollients, including, but not limited to: dimethicone cross-polymers, cyclomethicone, plant oils, polyisobutene, squalane, ceramides like lacto-ceramide, essential fatty acids (linoleic acid), fatty acids and esters of fatty alcohols and fatty acids, lanolin, lauric acids, stearic and palmitic acids with carbon chains lengths of 16 and 18 (coconut oil, grapeseed oil, and palm kernel oil), ceramides, combinations thereof, etc. In some embodiments proteins are provided that, like emollients, shrink on the skin (or a wound) leaving a film that smoothes the skin, thereby avoiding water loss (e.g., collagen, keratin, elastin, protein mixtures like wheat protein).

In various embodiments, a dispersion comprises one or more alcohols, including, but not limited to: acyclic alcohols (e.g., ethanol), isopropyl alcohol, etc.

In other embodiments, a dispersion comprises one or more adherents and/or film formers, including, but not limited to: trimethylsiloxysilicates, acrylates/dimethicones, etc.

In some embodiments, a dispersion comprises one or more conditioners, including, but not limited to: dimethicone gums, amine modified silicones, cetrimide, cetrimonium bromide, cetylamidopropyldimethyl ammonium chloride, combinations thereof, etc.

In particular embodiments, a dispersion comprises one or more preservatives, including, but not limited to: Phenonip, Parabens and esterof parabenzoic acids (phenoxyethanol), antioxidants (tocopherol, BHT, combinations thereof, etc.

In various embodiments, a dispersion comprises one or more oils and/or waxes, including, but not limited to: Aleurites moluccana seed oil, almond oil NF, anhydrous lanolin USP, apricot kernel oil, avocado oil, babassu oil, beeswax, borage seed oil, brazil nut oil, Cannibas sativa seed oil, canola oil, caprylic/capric triglyceride, carrot seed oil, ceresin, coconut oil, Daucus carota sativa root extract, dimethicone, dog rose hips oil, evening primrose oil, grape seed oil, hybrid safflower oil, jojoba oil, macadamia nut oil, Mangifera indica seed butter, olive oil, Oryza sativa oil, peanut oil NF, petrolatum, PPG-15 steryl ether, retinyl palmitate, sesame oil, soybean oil, sunflower oil, sweet almond oil, Theobroma cacao seed butter, tocopherol, combinations thereof, etc.

Chelating agents, e.g., a gluconate, may be used to chelate substances that may interfere with desired reactions and combinations, e.g., The chelating agent may be present to attract compounds that may interfere with the binding of the quaternary compound with the colloidal substrate Chelating agents are not usually required. A chelating agent, e.g., 20% of a 60% solution of gluconic acid and sodium gluconate as determined after neutralization, may be present.

In some embodiments, dispersions comprise or are added to animal feed (e.g., dry animal feed). In some embodiments, dispersions are combined with milk or milk substitutes or water.

In some embodiments, dispersions or suspensions for use in injection comprise pharmaceutically acceptable carriers. For example, compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Dispersions of the present disclosure provide significant advantages over administration of active agents via other carriers and/or systems, including, but not limited to: increased adsorption capacity for bacterial cells and toxins, reduction of toxicity of active ingredients, extended time of activity for active ingredients, time release characteristics, controlled release of active ingredients, ease of use, increased active ingredient loads of up to 200% of ion exchange capacity or greater, reduced irritation and enhanced effectiveness.

The methods of the present disclosure may include the steps of combining a hydrophilic clay in the form of particles having charged sites, with a compound having an ionic moiety and a hydrophobic tail to form a pre-combination and introducing the pre-combination into an aqueous phase to obtain an intermediate dispersion and combining a hydrophobic active compound with the intermediate dispersion to obtain further dispersion of particles having a substrate particle combined with an intermediate secondary layer having a hydrophobic tail and a tertiary layer including the active compound.

Further examples of detailed methods of making and using a dispersion of the disclosure include, but are not limited to the following. In certain embodiments the present disclosure provides methods of making a dispersion including one or more of the steps of: adding a humectant to deionized water to form an aqueous suspension; uniformly mixing a hydrophilic clay and a quaternary compound; adding water to the resulting clay and quaternary compound mixture to form a hydrophilic clay-quaternary ammonium compound combination; combining the aqueous suspension and the hydrophilic clay-quaternary ammonium compound combination to obtain a suspension; heating the suspension to between 70 and 90° C.; optionally dispersing together at least one active agent to obtain a dispersion; heating the dispersion to between 70 and 90° C.; mixing the dispersion with the suspension; drawing a vacuum; and homogenizing the resulting composition. In some embodiments the present disclosure provides methods of making a carrier system for a biologically active compound having a cationic moiety and a hydrophobic moiety including one or more of the steps of: mixing the biologically active compound and a hydrophilic clay to form an active compound-hydrophilic clay mixture; suspending the active compound-hydrophilic clay mixture in an aqueous liquid to form a suspension; and incorporating the suspension into a carrier. Any of the above or following methods may include adjusting pH if necessary, e.g., by adding KOH to mixed contents to adjust the pH to from about 5.2 to 6.2.

### Methods

The present disclosure finds use in a variety of applications and compositions. Exemplary applications and compositions are provided below. These should not be viewed as limiting: rather, the alterations and combinations of these embodiments are within the scope of the disclosure.

As described herein, in some embodiments, the compositions described herein find use in treating and preventing symptoms of scours in animal such as cattle (e.g., diarrhea and/or enteritis). In some embodiments, the cattle are calves. In some embodiments, treatment is initiated when the calves are 0 to 30 days old (e.g., 0, 1, 2, 3, 4, 5, 10, 20, or 30 days old). In some embodiments, the administering is repeated daily for 1 to 30 days (e.g., 1, 2, 3, 4, 5, 6, 7, 10, 14, or 16 days). In some embodiments, the administering is initiated within 2 to 12 hours after birth of the calve. In some embodiments, the administering is at least 2 hours after an initial colostrum feeding. In some embodiments, the administering is at least 2 hours after feeding the cattle. In some exemplary embodiments, the compositions are administered beginning the day of birth and continuing until the calf is 14-16 days old.

In some embodiments (e.g., when the gastrointestinal lining of the calf or adult cow is compromised by scours), bacteria (e.g., bacteria that are causing scours or other bacteria) can get into the bloodstream and cause sepsis. Accordingly, in some embodiments, dispersions described herein are administered in order to treat or prevent sepsis.

In some embodiments, the scours is bacterial. In some embodiments, the administration of dispersions of the present disclosure prevents the growth of or kill the bacteria or other microbial cause of scours. In some embodiments, death or morbidity of the calf is prevented.

In some embodiments, compositions are delivered to calves in liquid (e.g., milk, milk replacer, or water) or in animal feed. In some embodiments, compositions are delivered intravenously.

In some embodiments, 0.001-20 g (e.g., 0.01 to 10 g, 0.1 to 5 g, 1 to 2 g, etc.) of the dispersion (alone or in a formulation described herein) is administered 1-5 times daily for 1 to 30 days.

In some embodiments, such are provided in the form of a kit comprising the composition, including one or more active agents, and an administration component or device. In some embodiments, kits comprise a unit dose form of a composition.

### EXPERIMENTAL

### Example 1

### Combinations of Materials

TABLE 1
Hydrated Benzethonium Laponite-
Benzethonium Chloride Component Table.
In some embodiments, ingredients are for
pharmaceutical grade type products.

| Components | Ingredients | Function(s) |
|---|---|---|
| Purified Water | Purified Water | Continuous Phase |
| Benzethonium- | Laponite clay, | Sorbent & Thickener |
| Laponite | Benzethonium cation | |
| Benzethonium | Benzethonium | Sorbent & |
| Chloride | Chloride | Antimicrobial |

TABLE 2
Hydrated Cetylpyridinium Hectorite-
Cetylpyridinium Chloride Component Table.
In some embodiments, ingredients
are for higher end animal feeds.

| Components | Ingredients | Function(s) |
|---|---|---|
| Purified Water | Purified Water | Continuous Phase |
| Cetylpyridinium- | Hectorite clay, | Sorbent & Thickener |
| Hectorite | Cetylpyridinium | |
| | cation | |
| Cetylpyridinium | Cetylpyridinium | Sorbent & |
| chloride | chloride | Antimicrobial |

TABLE 3
Hydrated Cetylpyridinium Montmorillonite-
Cetylpyridinium Chloride Component Table.
In some embodiments, ingredients
are for lower end animal feeds.

| Components | Ingredients | Function(s) |
|---|---|---|
| Purified Water | Purified Water | Continuous Phase |
| Cetylpyridinium- | Montmorillonite clay, | Sorbent & |
| Montmorillonite | Cetylpyridinium | Thickener |
| | cation | |
| Cetylpyridinium | Cetylpyridinium | Sorbent & |
| Chloride | chloride | Antimicrobial |

### Methods of Manufacture

### Lab Scale Process

1. Mix clay and quaternary ammonium compound (quat) together, creating a dry mix. Add enough quat to clay to satisfy 2.0 or more of the cation exchange capacity (C.E.C.) of the clay by dry weight.
2. Hydrate the dry mix with water (purified and at 60° C. preferred) to allow continuous/fluid movement.
3. Allow to hydrate/mix overnight (maintaining heat if possible) in an incubator shaker.
4. Mix well before placing into lab scale centrifuge at 3500 rpm for 15 minutes.
5. Pour off decant and add water (purified and at 60° C. preferred) to wash material (wash 1).
6. Repeat steps 4 and 5 (wash 2).
7. Store material for later use.

Note: This process produces clays with water content between 60% and 70%.

### Pilot Scale Process

### Decanter Centrifuge

1. Mix clay and quaternary ammonium compound (quat) together in rotary mixer for 15 minutes. Add enough quat to clay to satisfy 2.0 or more of the cation exchange capacity (C.E.C.) of the clay by dry weight.
2. Transfer dry mix to a slurry mixing tank and add water (purified and at 60° C. preferred) to allow continuous/fluid movement.
3. A defoamer may be added if deemed necessary.
4. Filter material of excess water and quat using a continuous decanter centrifuge.
5. Store material for later use.

Note: This process produces clays with water content between 50% and 70%

### Filter Press

1. Mix clay and quaternary ammonium compound (quat) together in rotary mixer for 15 minutes. Add enough quat to clay to satisfy 2.0 or more of the cation exchange capacity (C.E.C.) of the clay by dry weight.
2. Transfer dry mix to a slurry mixing tank and add water (purified and at 60° C. preferred) to allow continuous/fluid movement.
3. A defoamer may be added if deemed necessary.
4. Filter material of excess water and quat using a manual/automatic filter press.
5. Continue to air dry until desired water content is reached.
6. Store material for later use.

Note: This process produces clays with water content between 1% to 55%.

### Formulations

### Exemplary Formulation 1: Gelatin-Syringe Formulation

TABLE 4
The Gelatin Syringe Formulation Component
Table. The highly loaded Organoclay
may be switched out for another.
^{a}Concentration in formulation is
dependent on the available pat.

| | | Concentration % |
|---|---|---|
| Components | Function(s) | (w/w) |
| Hydrated Benzethonium | Antimicrobial | 7.6^{a} |
| Laponite-Benzethonium | Sorbent | |
| Chloride | | |
| Purified Water | Water Phase | q.s. |
| Gelatin Powder | Thickening Agent | 1.0-2.5 |

### Exemplary Formulation 2: Gummy Formulation

TABLE 5
Gummy Formulation Component Table.
The highly loaded Organoclay may
be switched out for another.
^{a}Concentration in formulation is
dependent on the available quat.

| | | Concentration % |
|---|---|---|
| Components | Function(s) | (w/w) |
| Hydrated Benzethonium | Antimicrobial | 7.6^{a} |
| Laponite-Benzethonium | Sorbent | |
| Chloride | | |
| Purified Water | Water Phase | q.s. |
| Sugar Free Gelatin | Thickening | 3.5 |
| Dessert Powder | Agent & | |
| | Flavor | |
| Gelatin Dessert Powder | Thickening | 45.90 |
| | Agent & | |
| | Flavor | |

### Gelatin Syringe & Gummy Formulation Steps

1. Add all gelatin powder to beaker A.
2. To beaker B, add the highly loaded organoclay and water and mix until dispersed.
3. Homogenize, then under stir, heat contents of beaker B until 70° C. to 80° C.
4. Pour contents of beaker B into beaker A, heating and mixing contents.
5. Pour 60° C. mixture into syringe/mold, followed by refrigeration at 4-6° C. to allow to set.

### Exemplary Formulation 3: No Additives Syringe Formulation

TABLE 5
No Additives Syringe Formulation.
The highly loaded Organoclay
may be switched out for another.

| Components | Function(s) | Amount | |
|---|---|---|---|
| Hydrated Benzethonium | Antimicrobial | 4 | g |
| Laponite-Benzethonium | Sorbent | | |
| Chloride | | | |
| Purified Water | Water Phase | 30 | ml |

No Additives Syringe Formulation Steps
1. Remove plunger from sterile syringe.
2. Load highly loaded organoclay into the cavity of the syringe, the remove air by re-inserting plunger.
3. Seal end with syringe cap and vacuum seal external package.

Directions for Use
1. Remove syringe from pack.
2. Take up water to designated marking on plunger (30 ml).
3. Invert syringe and pull to fill with air.
4. Shake vigorously to disperse product in water.
5. Administer orally as per recommendation.

### Exemplary Formulation 4: Flavored Grain Ball Formulation (for Horses)

TABLE 5
Flavored Grain Ball Formulation Component Table.
The highly loaded Organoclay may be switched
out for another, as well as the type of feed and flavor.

| Components | Function(s) | Amount |
|---|---|---|
| Hydrated Benzethonium | Antimicrobial Sorbent | 40 g |
| Laponite-Benzethonium | | |
| Chloride | | |
| Dry Animal Feed: | Vehicle | q.s. |
| Alfalfa Hay | | |
| & Processed | | |
| Grain Mixture. | | |
| Carrot, Apple, or Molasses | Flavor & Sweetener | TBD |
| Molasses | Thickener & Sweetener | TBD |

Flavored Grain Ball Formulation Steps

1. Mix ground carrot together with the highly loaded organoclay.

2. Add the dry animal feed and molasses to the mixture and mix with kitchen aid.

3. Knead dough-like material into a ball.

4. Vacuum pack and refrigerate.

### Exemplary Formulation 5: Animal Feed Additive/Supplement

The highly loaded organoclay is readily added to milk replacer, water supply or dry animal feed. In some embodiments, 1-20 g of the dispersion (alone or in a formulation described herein) is administered 1-5 times daily for 1 to 30 days.
The highly loaded organoclay with a high and low water content may be used for this application.

### Indications

In some embodiments, the compositions described herein find use to prevent or treat enteric disease in livestock by killing and removing bacteria and bacterial toxins associated with enteric disease.

### Directions for Use

As a prophylactic to newborns, administer 2 to 12 hours after birth, and at least 2 hours after initial colostrum feeding for optimal efficacy.
For treatment purposes, administer at least 2 hours after feeding for optimal efficacy.

### Example 2

### Pharmacokinetic (PK) Study

a. Study Design:
   (1) Objective: To confirm Busumite™ to be non-systemic by conducting a pharmacokinetic study over the course of 3 days after administering 5× the normal dose.
   (2) The study utilized a laponite/benzethonium chloride dispersion (BC200). BC200 was prepared by the following method:
      1 6.3 g of Benzethonium Chloride (BTC) was added to a 1 L bottle.
      2 463.0 g of deionized (DI) water was added to the bottle, then mixed to dissolve the BTC.
      3 25.0 g of LAPONITE RDS was added to the bottle and mixed.
      4 The bottle was placed into 60 C oven for 24 hours.
      5 The bottle was centrifuged for 5 minutes at 3500 rpms and the supernant decanted.
      6 A second solution of BTC (6.3 g of BTC and 463.0 g of DI) was added and mixed.
      7 The bottle was placed back into th 60 C oven for 24 hours.
      8 The bottle was centrifuged for 5 minutes at 3500 rpms and the supernant decanted.
      9 A third solution of BTC (6.3 g of BTC and 463.0 g of DI) was added and mixed.
      10 The bottle was placed back into th 60 C oven for 24 hours.
      11 The bottle was centrifuged for 5 minutes at 3500 rpms and the supernant decanted.
      12 463.0 g of DI water was added to the bottle, then mixed. This is wash 1.
      13 The bottle was centrifuged for 5 minutes at 3500 rpms and the supernant decanted.
      14 Steps 12 and 13 were repeated twice more. This is wash 2 and 3.
   (3) Test Animal: One 2-week old, healthy, Holstein calf.
   (4) Test Article Administration: The test article was housed in two (2) injectable 30 ml syringe, containing 20 grams of Busumite™, containing 20 mg of BZT per kg. The test article and made into solution using tap water and mixed before administration.
   (5) Measurements and Observations: The animal was observed twice per day for signs of adverse effects.
   (6) Sample Collection and Analysis: Blood was drawn at various time points (Before administration, 4, 12, 24, 48, and 72 hours) over the course of 3 days. Samples were processed, stored, and transported, and logged in accordance to protocol. Further extraction and analysis was performed in accordance with validated LC method for the recovery of Benzethonium chloride (BZT) from bovine calf blood plasma.
b. Results:
   Benzethonium Chloride Plasma Analysis: There was no detectable levels of Benzethonium chloride in the blood at any of the time points. Tests were conducted using proper controls.
c. Adverse Reactions: There were no adverse reactions.
d. Conclusions: Following the oral administration of Busumite™ suspension at 5× normal dose, 20 g Busumite™, 333.33 mg/kg, equivalent to 20 mg Benzethonium chloride/kg, analysis of blood plasma suggests no delivery into systemic circulation.

### Example 3

Efficacy Study

### BACKGROUND

This study tests the administration of Busumite within 4-12 hours after birth to reduce the incidence of scours and aid the calf's immune system during the most critical time immediately after birth (0-72 hours of life).
e. Study Design:
   (1) Objective: To determine the utility of Busumite™ in the prevention of calf scours.
   (2) Test Animal: Forty (40) neonatal Holstein calves, originating from a dairy farm.
   (3) Study Design: This is a placebo controlled randomized blind study. Twenty (20) calves in each treatment group received an oral dosage via syringe on the first day of life. Colostrum was given within 2 hours of birth and the test article(s) was administered at least 4-12 hours after birth and 2-4 hours after initial feeding. The in-life phase study was 28 days.
   (4) Test Article Administration: The test article was housed in one (1) injectable 30 ml syringe, containing 4 grams of Busumite™, containing 6.86 mg of BZT per kg. The test article made into solution using tap water to serve as a vehicle and mixed before administration. Commercially available milk replacer was used as the placebo.
   (7) Measurements and Observations: Body weights were taken before and after treatment. The animals were checked once daily as defined by vet or study monitor for clinical signs (appearance of fecal matter abnormality, drinking, up and active or not, abdominal bloating, coat characteristics).
   (8) Clinical Laboratory Studies and Pathology: Hematology and serum chemistry were performed on all calves at various time points. Necropsy and histology were performed on animals found in moribund condition, at the discretion of the veterinarian, or if the animal died in the study.
f. Results:

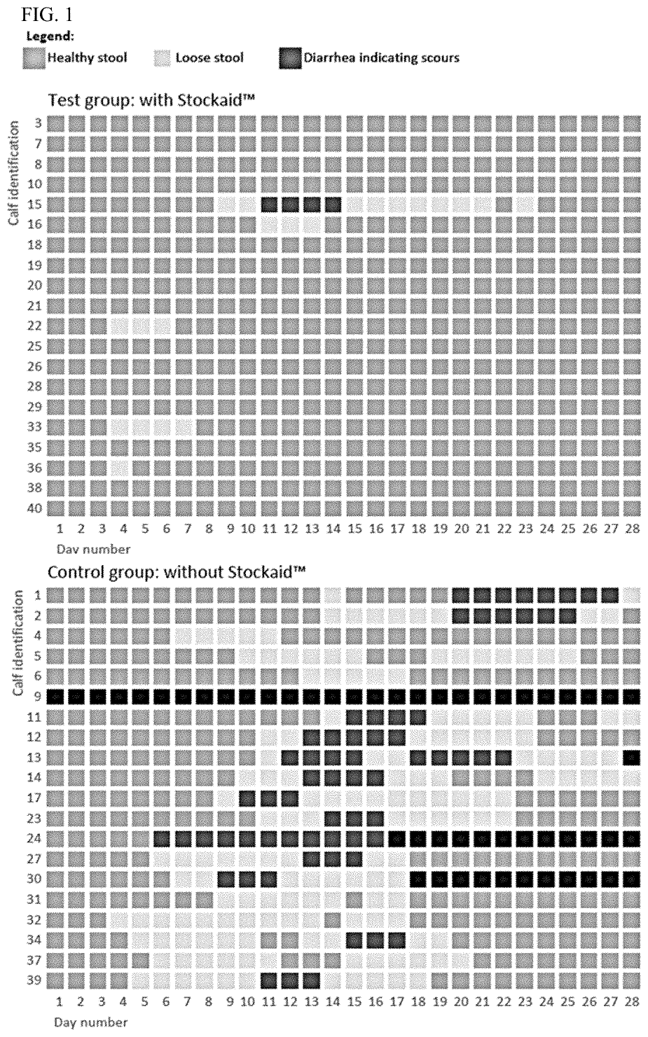

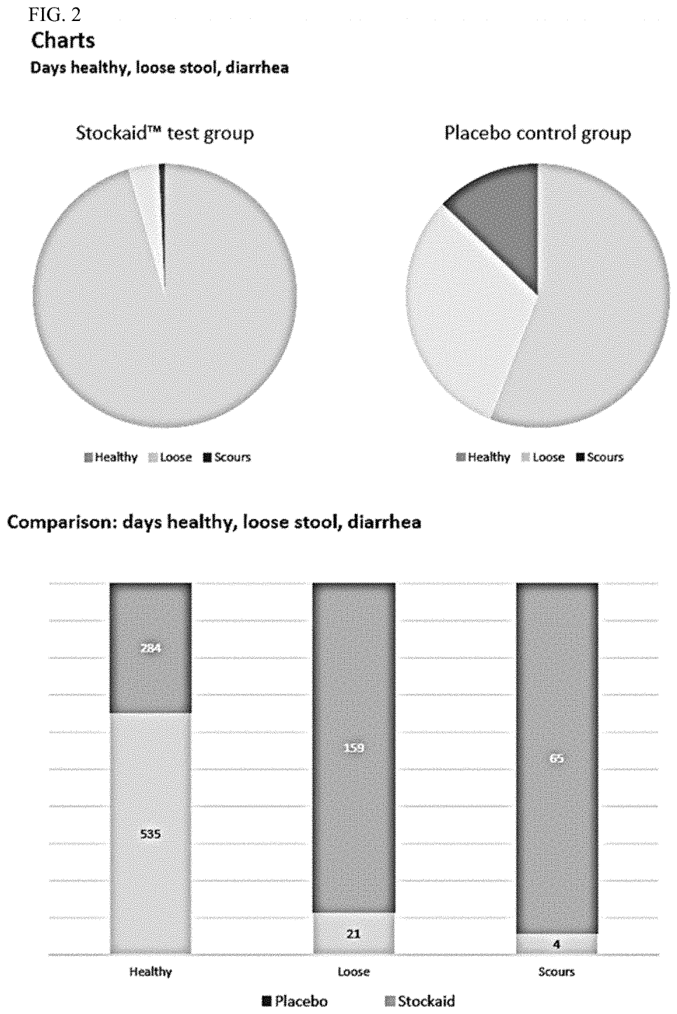

The incidence of scours was 65% for the placebo group compared to 5% of the Busumite™ group. In addition, 20% of the calves from the placebo group died due to complications related to the onset of enteric disease associated with scours. There were no deaths in the Busumite™ treatment group. Results are shown in FIGS. 1-2 and Table 6.

TABLE 6
Comparative analysis:

| | Test herd | Control |
|---|---|---|
| | (Stockaid**) | herd (placebo) |
| Mortality number | 0 | 4 |
| Mortality % | 0.0% | 20% |
| Incidence of scours | 1 | 13 |
| Incidence of scours % | 5.0% | 65.0% |
| Days of illness | 25 | 224 |
| Days of illness % | 4.5% | 40.0% |

It is to be understood that this disclosure is not limited to the particular methodology, materials and modifications described and as such may, of course, vary. It is also understood that the terminology used herein is for the purpose of describing particular aspects only, and is not intended to limit the scope of the present disclosure, which is limited only by the appended claims. Although any methods, devices or materials similar or equivalent to those described herein can be used in the practice or testing of the disclosure, they should not be viewed as limiting.

1. A method of treating or preventing enteritis and/or diarrhea in an animal, comprising:
administering a composition comprising a dispersion to said cattle, said dispersion comprising a dispersed phase within an aqueous phase, and wherein the dispersion comprises:
a) a hydrophilic clay in said dispersed phase; and
b) a quaternary ammonium compound in said dispersed phase,
wherein said administering treats or prevents said enteritis and/or diarrhea in said animal.

2. The method of claim 1, wherein said hydrophilic clay comprises one or more of smectite, laponite, hectorite, montmorillonite, and bentonite clays.

3. The method of claim 1, wherein said quaternary ammonium compound comprises benzethonium chloride, cetylpyridinium chloride, or derivatives thereof.

4. The method of claim 1, wherein said animal is cattle or a calf.

5. The method of claim 4, wherein said calves are 0 to 30 days old.

6. The method of claim 1, wherein said administering is repeated daily for 1 to 30 days.

7. The method of claim 1, wherein said administering is initiated within 2 to 12 hours after birth of said animal.

8. The method of claim 7, wherein said administering is at least 2 hours after an initial colostrum feeding.

9. The method of claim 1, wherein said administering is at least 2 hours after feeding said animal.

10. The method of claim 1, wherein said dispersion is combine with one or more of a milk replacer, water, and dry animal feed.

11. The method of claim 1, wherein said administration is intravenous administration.

12. The method of claim 1, wherein said dispersion further comprises an active agent, wherein said active agent comprises one or more of: an antimicrobial, an analgesic, an anti-fungal, and an anti-inflammatory.

13. (canceled)

14. The method of claim 1, wherein said dispersion comprises one or more of a chelating agent, emulsifier, humectants, moisturizer, detackifier, emollient, animal feed, flavoring agent, and thickener.

15. The method of claim 1, wherein said dispersion is made by the method of i) dry mixing said hydrophilic clay and said quaternary ammonium compound to produce a mixture; ii) adding water to said mixture; and iii) incubating said mixture to form said dispersion.

16. The method of claim 1, wherein said enteritis and/or diarrhea is caused by a bacterial infection.

17. The method of claim 16, wherein said administration kills or prevent the growth of the bacteria and/or neutralizes a toxin secreted by the bacteria.

18. The method of claim 1, wherein said administering prevents death of the animal.

19. The method of claim 1, wherein said composition comprises 0.001 g to 20 g of dispersion per dose.

20. (canceled)

21. A composition comprising a dispersion, said dispersion comprising a dispersed phase within an aqueous phase, and wherein the dispersion comprises:
a) a hydrophilic clay in said dispersed phase; and
b) a quaternary ammonium compound in said dispersed phase,
for use to treat or prevent enteritis and/or diarrhea in an animal.

22. A method of treating or preventing enteritis and/or diarrhea in a calf, comprising: administering a composition comprising a dispersion to said cattle, said dispersion comprising a dispersed phase within an aqueous phase, and wherein the dispersion comprises:
a) a hydrophilic clay in said dispersed phase; and
b) a quaternary ammonium compound in said dispersed phase,
wherein said administering treats or prevents said enteritis and/or diarrhea in said calf.
